(19) United States
(12) Patent Application Publication
(10) Pub. No.: US 2005/0064542 A1
(43) Pub. Date: Mar. 24, 2005

(54) Nucleic acid expressed in the hypothalamus or muscle tissue in obese animals

(76) Inventors: Greg Collier, Ocean Grove (AU); Ken Walder, Ocean Grove (AU); Janine McMillian, Torquay (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/467,606

(22) PCT Filed: Feb. 5, 2002
(86) PCT No.: PCT/AU02/00109

(30) Foreign Application Priority Data
Feb. 7, 2001 (AU) PR 2950

(51) Int. Cl. C07H 21/04; C07K 14/575

(52) U.S. Cl. 435/69.1; 435/320.1; 435/325; 530/399; 536/23.5

(57) ABSTRACT

An isolated nucleic acid molecule comprising a sequence of nucleotides encoding or complimentary to a sequence encoding a molecule or derivative or homologue thereof wherein said nucleic acid molecule is expressed in a larger amount in one or both hypothalamus tissue or muscle tissue of obese animals compared to lean animals or in fed animals compared to fasted animals. Nucleic acid sequences are disclosed. It is proposed to use the expression products of such nucleic acids as modulators and/or monitors of physiological processes associated with obesity, anorexia, weight maintenance, impaired muscle development, diabetes and/or metabolic energy levels.

### FIELD OF THE INVENTION

The present invention relates generally to nucleic acid molecules expressed in hypothalamus or skeletal muscle tissue and identified using a differential display or macroarray technique or other techniques capable of detecting differential expression of nucleic acid molecules under differing physiological conditions. Expression products from the nucleic acid molecules of the present invention are associated with or act as markers for one or more of a healthy state, obesity, anorexia, weight maintenance, impaired muscle development, diabetes and/or metabolic energy levels and/or altered physiological conditions. The identification of the present nucleic acid molecules and their expression products and/or their derivatives, homologs, analogs and mimetics are proposed to be useful as therapeutic and diagnostic agents or as targets for agents which act as modulators and/or monitors of physiological processes associated with obesity, anorexia, weight maintenance, impaired muscle development, diabetes and/or metabolic energy levels and/or other physiological conditions.

### BACKGROUND OF THE INVENTION

Reference to any prior art in this specification is not, and should not be taken as, an acknowledgment or any form of suggestion that this prior art forms part of the common general knowledge in Australia or any other country.

The increasing sophistication of recombinant DNA technology is greatly facilitating research and development in the veterinary and allied human and animal health fields. This is particularly the case in the investigation of the genetic bases involved in the etiology of certain disease conditions. One particularly significant condition from the stand point of morbidity and mortality is obesity and its association with type 2 diabetes (formerly non-insulin-dependent diabetes mellitus or NIDDM) and cardiovascular disease.

Obesity is defined as a pathological excess of body fat and is the result of an imbalance between energy intake and energy expenditure for a sustained period of time. Obesity is the most common metabolic disease found in affluent nations. The prevalence of obesity in these nations is alarmingly high, ranging from 10% to upwards of 50% in some subpopulations (Bouchard, The genetics of obesity. Boca Raton: CRC Press, 1994). Of particular concern is the fact that the prevalence of obesity appears to be rising consistently in affluent societies and is now increasing rapidly in less prosperous nations as they become more affluent and/or adopt cultural practices from the more affluent countries (Zimmet, Diabetes Care 15(2): 232-247, 1992).

In 1995 in Australia, for example, 19% of the adult population were obese (BMI>30). On average, women in 1995 weighed 4.8 kg more than their counterparts in 1980 while men weighed 3.6 kg more (Australian Institute of Health and Welfare (AIHW), Heart, Stroke and Vascular diseases, Australian facts. AIHW Cat. No. CVD 7 Canberra: AIHW and the Heart Foundation of Australia, 1999.). More recently, the AusDiab Study conducted between the years 1999 and 2000 showed that 65% of males and 45% of females aged 25-64 years were considered overweight (de Looper and Bhatia, Australia's Health Trends 2001. Australian Institute of Health and Welfare (AIHW) Cat. No. PHE 24. Canberra: AIHW, 2001). The prevalence of obesity in the US also increased substantially between 1991 and 1998, rising from 12% to 18% in Americans during this period (Mokdad et al., JAMA. 282(16): 1519-22, 1999).

The high and increasing prevalence of obesity has serious health implications for both individuals and society as a whole. Obesity is a complex and heterogeneous disorder and has been identified as a key risk indicator of preventable morbidity and mortality since obesity increases the risk of a number of other metabolic conditions including type 2 diabetes mellitus and cardiovascular disease (Must et al., JAMA. 282(16): 1523-1529, 1999; Kopelman, Nature 404: 635-643, 2000). Alongside obesity, the prevalence of diabetes continues to increase rapidly. It has been estimated that there were about 700,000 persons with diabetes in Australia in 1995 while in the US, diabetes prevalence increased from 4.9% in 1990 to 6.9% in 1999 (Mokdad, Diabetes Care 24(2): 412, 2001). In

Australia, the annual costs of obesity associated with diabetes and other disease conditions has been conservatively estimated to be AU$810 million for 1992-3 (National Health and Medical Research Council, Acting on Australia's weight: A strategy for the prevention of overweight and obesity. Canberra: National Health and Medical Research Council, 1996). In the US, the National Health Interview Survey (NHIS) estimated the economic cost of obesity in 1995 as approximately US$99 billion, thereby representing 5.7% of total health costs in the U.S. at that time (Wolf and Colditz, Obes Res. 6: 97-106, 1998).

A genetic basis for the etiology of obesity is indicated inter alia from studies in twins, adoption studies and population-based analyses which suggest that genetic effects account for 25-80% of the variation in body weight in the general population (Bouchard [1994; supra]; Kopelman et al., Int J Obesity 18: 188-191, 1994; Ravussin, Metabolism 44(Suppl 3): 12-14, 1995). It is considered that genes determine the possible range of body weight in an individual and then the environment influences the point within this range where the individual is located at any given time (Bouchard [1994; supra]). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

A number of organs/tissues have been implicated in the pathophysiology of obesity and type 2 diabetes, and of particular interest is the hypothalamus. The hypothalamus has long been recognized as a key brain area in the regulation of energy intake (Stellar, Psychol Rev 61: 5-22, 1954) and it is now widely accepted that the hypothalamus plays a central role in energy homeostasis, integrating and co-ordinating a large number of factors produced by and/or acting on the hypothalamus. A number of these factors have been investigated for their role in energy balance and body weight regulation, including neuropeptide Y, corticotropin-releasing hormone, melanin-concentrating hormone, leptin and insulin. It has been proposed that genetic alterations perturbing the metabolic pathways regulating energy balance in the hypothalamus could contribute to the development of obesity, and subsequently diabetes. Thus, an important step in understanding the function of the hypothalamus in regulating the metabolism of an animal requires the identification of the targets of these hormones. Such targets may be whole organs, and genes whose expression is regulated by the presence of these hormones.

In accordance with the present invention, the subject inventors sought to identify genetic sequences which are differentially expressed in lean and obese animals or in fed compared to unfed animals. Using techniques such as differential display and macroarray (i.e. membrane-based microarray) analysis, the inventors identified genes which are proposed to be associated with one or more biological functions connected with a healthy state or a disease condition such as but not limited to obesity, anorexia, weight maintenance, diabetes, muscle development and/or metabolic energy levels and/or other altered physiological conditions.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

Techniques including differential display analysis and macroarray (i.e. membrane-based microarray) analysis of genetic material from hypothalamus tissue or muscle tissue were used to identify candidate genetic sequences associated with a healthy state or with physiological conditions such as obesity, anorexia, weight maintenance, diabetes, muscle development and/or metabolic energy levels. An animal model was employed comprising the Israeli Sand Rat (Psammomys obesus). Three groups of animals are used designated Groups A, B and C based on metabolic phenotype as follows:—
Group A: lean animals;
Group B: obese, non-diabetic animals; and
Group C: obese, diabetic animals.

Animals were maintained under fed or unfed conditions or under conditions of high or low glucose or insulin and genetic sequences analyzed by differential display and macroarray analysis. In a preferred embodiment using these techniques, four putatively differentially expressed sequences were identified from hypothalamus cells designated herein AGT-106, AGT-113, AGT-201 and AGT-202 with sequence identifiers SEQ ID NO:1, SEQ ID NO:2, SEQ ID NO:3, SEQ ID NO:4, respectively. Another give, AGT-203 (SEQ ID NO:5) was differentially expressed in skeletal muscle.

Differential expression means an elevation in levels of expression of a genetic sequence under one set of conditions compared to another. In one particular embodiment, AGT-106 expression was elevated in fed animals versus lean animals. In obese animals, AGT-106 was found to be suppressed even when fed. AGT-106 is involved in regulation of energy utiliation and body weight in response to fasting. AGT-113 is expressed higher in diabetic, obese animals relative to lean, healthy animals indicating that AGT-113 is involved in body weight regulation and energy homeostasis and may also be involved in the action of insulin or insulin resistance in the hypothalamus. AGT-201 was identified using macroanalysis and its expression was lower in fasted animals. AGT-201 is involved in the central response to fasting and energy homeostasis. AGT-201 may also have a role in diabetes. AGT-202 are also identified by macroanalysis and was shown to be elevated in the hypothalamus of fed animals compared to fasted animals and is likely to be involved in energy regulation and/or body weight maintenance. Finally, AGT-203 was differentially expressed in skeletal muscle of lean, non-diabetic versus obese, diabetic animals using macroarray analysis. It is likely that AGT-203 plays a role in glucose or fat metabolism in skeletal muscle, thereby affecting body weight and insulin action. A summary of the AGT sequences is provided in Table 1.

The identification of these variably expressed sequences permits the rationale design and/or selection of molecules capable of antagonizing or agonizing the expression products and/or permits the development of screening assays. The screening assays, for example, include assessing the physiological status of a particular subject.

Accordingly, one aspect of the present invention provides a nucleic acid molecule comprising a sequence of nucleotides encoding or complementary to a sequence encoding a protein or a derivative, homolog, analog or mimetic thereof wherein the nucleic acid molecule is expressed in larger amounts in hypothalamus or muscle tissue of obese animals compared to lean animals. Alternatively, or in addition, the nucleic acid molecule is expressed in larger amounts in hypothalamus or muscle tissue of fed animals compared to fasted animals.

In a preferred embodiment, the nucleic acid molecule comprises a nucleotide sequence substantially as set forth in SEQ ID NO:1 or SEQ ID NO:2 or SEQ ID NO:3 or SEQ ID NO:4 or SEQ ID NO:5 or a nucleotide sequence having at least about 30% similarity to all or part of SEQ ID NO: 1 or SEQ ID NO:2 or SEQ ID NO:3 or SEQ ID NO:4 or SEQ ID NO:5 and/or is capable of hybridizing to one or more of SEQ ID NO: 1 or SEQ ID NO:2 or SEQ ID NO:3 or SEQ ID NO:4 or SEQ ID NO:5 or their complementary forms under low stringency conditions at 42° C.

Another aspect of the present invention provides an isolated molecule or a derivative, homolog, analog or mimetic thereof which is produced in a larger amount in hypothalamus tissue of obese animals compared to lean animals and/or which is produced in a larger amount in hypothalamus tissue of fed animals compared to fasted animals.

The molecule is generally a protein but may also be an mRNA, intron or exon.

The molecule is encoded by a nucleotide sequence substantially as set forth in SEQ ID NO:1, SEQ ID NO:2, SEQ ID NO:3, SEQ ID NO:4 or SEQ ID NO:5 or a nucleotide sequence having at least 30% similarity to all or part of SEQ ID NO:1, SEQ ID NO:2, SEQ ID NO:3, SEQ ID NO:4 or SEQ ID NO:5 and/or is capable of hybridizing to SEQ ID NO:1, SEQ ID NO:2, SEQ ID NO:3, SEQ ID NO:4 or SEQ ID NO:5 under low stringency conditions at 42° C.

In this respect, the molecule may be considered an expression product of the subject nucleotide sequences.

The preferred genetic sequences of the present invention are referred to herein as AGT-106, AGT-113, AGT-201, AGT-202 and AGT-203. The expression product encoded by AGT-106, AGT-113, AGT-201, AGT-202 and AGT-203 are referred to herein as AGT-106, AGT-113, AGT-201, AGT-202 and AGT-203, respectively. The preferred expression products are proteins.

A further aspect of the present invention relates to a composition comprising an expression product such as a protein defined by AGT-106, AGT-113, AGT-201, AGT-202 and AGT-203 or its derivatives, homologs, analogs or mimetics or agonists or antagonists of AGT-106, AGT-113, AGT-201, AGT-202 and AGT-203 together with one or more pharmaceutically acceptable carriers and/or diluents.

Furthermore, the present invention contemplates a method for treating a subject comprising administering to the subject, a treatment effective amount of AGT-106, AGT-113, AGT-201, AGT-202 and AGT-203 or a derivative, homolog, analog or mimetic thereof or a genetic sequence encoding same or an agonist or antagonist of AGT-106, AGT-113, AGT-201, AGT-202 and AGT-203 or AGT-106, AGT-113, AGT-201, AGT-202 and AGT-203 gene expression for a time and under conditions sufficient to effect treatment.

In accordance with this and other aspects of the present invention, treatments contemplated herein include but are not limited to obesity, anorexia, weight maintenance, energy imbalance and diabetes. Treatment may be by the administration of a pharmaceutical composition or genetic sequences via gene therapy. Treatment is contemplated for human subjects as well as animals such as animals important to livestock industry.

Still another aspect of the present invention is directed to a diagnostic agent for use in monitoring or diagnosing conditions such as but not limited to obesity, anorexia, weight maintenance, energy imbalance and/or diabetes, said diagnostic agent selected from an antibody to AGT-106, AGT-113, AGT-201, AGT-202 and AGT-203 or its derivatives, homologs, analogs or mimetics and a genetic sequence useful in PCR, hybridization, RFLP amongst other techniques.

A summary of sequence identifiers used throughout the subject specification is provided in Table 2.

TABLE 1
Summary of Differentially Expressed Genes

| | SEQ ID | | | METHOD OF |
|---|---|---|---|---|
| GENE | NO: | TISSUE | PHENOTYPE | DETECTION |
| AGT-106 | 1 | Hypothalamus | Higher expression in fed | Differential |
| | | | compared to fasted Sand | display |
| | | | rats (Group A [lean, | |
| | | | healthy rats]) | |
| AGT-113 | 2 | Hypothalamus | Higher expression in | Differential |
| | | | Group C (obese, diabetic) | display |
| | | | compared to Group A | |
| AGT-201 | 3 | Hypothalamus | Lower expression in fasted | Macroarray |
| | | | A and B (obese, | |
| | | | normoglycemic and hyerinsulinemic) | |
| | | | animals | |
| AGT-202 | 4 | Hypothalamus | Higher expression in fed | Differential |
| | | | compared to fasted | display |
| | | | animals in Group A and B | |
| | | | compared to Group C | |
| | | | animals | |
| AGT-203 | 5 | Skeletal muscle | Higher expression in lean, | Macroarray |
| | | | non-diabetic (Group A) | |
| | | | animals compared to | |
| | | | (obese, diabetic) Group C | |

TABLE 2
Summary of Sequence Identifiers

| SEQUENCE ID NO. | DESCRIPTION |
|---|---|
| 1 | Nucleotide sequence of AGT-106 |
| 2 | Nucleotide sequence of AGT-113 |
| 3 | Nucleotide sequence of AGT-201 |
| 4 | Nucleotide sequence of AGT-202 |
| 5 | Nucleotide sequence of Presenilins interacting |
| | rhomboid-like protein (AGT-203) |
| 6 | AGT-203 forward primer |
| 7 | AGT-203 reverse primer |
| 8 | AGT-203 probe |
| 9 | AGT-201 forward primer |
| 10 | AGT-201 reverse primer |
| 11 | AGT-201 probe |
| 12 | AGT-106 forward primer |
| 13 | AGT-106 reverse primer |
| 14 | AGT-202 forward primer |
| 15 | AGT-202 reverse primer |
| 16 | AGT-113 forward primer |
| 17 | AGT-113 reverse primer |
| 18 | Cyclophilin forward primer |
| 19 | Cyclophilin reverse primer |
| 20 | Cyclophilin probe |

### BRIEF DESCRIPTION OF THE DRAWINGS

FIG. 1 is a graphical representation of AGT-106 gene expression in hypothalamus fed and fasted groups of Psammomys obesus animals. In the hypothalamus, AGT-106 expression was decreased with fasting in group A animals and remained unchanged with fasting in group B and C animals. Although the decrease with fasting in group A animals represented a 55% reduction this did not reach significance when compared by ANOVA with a Games-Howell post-hoc test.

FIG. 2 is a graphical representation of AGT-106 gene expression in hypothalamus in a fed and fasted Psammomys obesus animals. AGT-106 expression was significantly decreased with fasting when all animals were combined (p=0.035).

FIG. 3 is a graphical representation of AGT-106 gene expression versus body weight of fasted Psammomys obesus animals. In the hypothalamus, AGT-106 expression significantly, negatively correlated with the change in bodyweight after a 24-hour fast (R=0.483, p=0.023, all fasted animals).

FIG. 4 is a graphical representation of AGT-106 gene expression versus body weight in fasted lean Psammomys obesus animals. In lean, group A animals, hypothalamic AGT-106 expression was not associated with the change in bodyweight after a 24-hour fast.

FIG. 5 is a graphical representation of AGT-106 gene expression versus body weight in fasted obese Psammomys obesus animals. In obese, group B and C animals, hypothalamic AGT-106 expression was significantly, negatively associated with the change in bodyweight after a 24-hour fast (R=0.678, p=0.005).

FIG. 6 is a graphical representation of AGT-113 gene expression in hypothalamus of fed and fasted Group A, B and C Psammomys obesus animals.

FIG. 7 is a graphical representation of the correlation of AGT-113 gene expression in the hypothalamus with (A) body weight (fed animals); (B) percent body fat (fed animals); (C) plasma insulin (fed animals); and (D) body weight (fasted animals).

FIG. 8 is a graphical representation of gene expression of AGT-201 in hypothalamus in fed and fasted Psammomys obesus animals.

FIG. 9 is a graphical representation of expression of AGT-201 in hypothalamus of fed and fasted Psammomys obesus animals.

FIG. 10 is a graphical representation of expression of the AGT-202 gene in hypothalamus of fed and fasted Psammomys obesus animals.

FIG. 11 is a graphical representation of expression of the AGT-202 gene in the hypothalamus of fed and fasted Psammomys obesus animals.

FIG. 12 is a graphical representation showing (A) AGT-203 gene expression in the red gastrocinemius muscle of group A, B and C Psammonys obesus. (B) correlation of AGT-203 gene expression in the red gastrocinemius muscle with plasma insulin level; (C) correlation of AGT-203 gene expression in the red gastrocinemius muscle with body weight and (D) correlation of AGT-203 gene expression in the red gastrocinemius muscle with percent body weight.

### DETAILED DESCRIPTION OF THE PREFERRED EMBODIMENTS

The present invention is predicated in part on the identification of novel genes associated inter alia with regulation of energy balance obesity and diabetes and/or muscle development. The genes were identified by a number of procedures including differential screening or macroarray analysis of hypothalamus or skeletal muscle mRNA between lean and obese animals and/or between fed animals and fasted animals.

The term “differential” array is used in its broadest sense to include the expression of nucleic acid sequences in one type of tissue relative to another type of tissue in the same or different animals. Reference to “different” animals include the same animals but in different gastronomical states such as in a fed or non-fed state. Macroarray (i.e. membrane-based microarray) analysis preferably includes sets of arrays of nucleic and expression products (e.g. mRNA or PCR products) which display differential hybridization characteristics.

Accordingly, one aspect of the present invention provides a nucleic acid molecule comprising a sequence of nucleotides encoding or complementary to a sequence encoding an expression product or a derivative, homolog, analog or mimetic thereof wherein said nucleic acid molecule is expressed in larger amounts in hypothalamus or muscle tissue of obese animals compared to lean animals.

In a related embodiment, there is provided a nucleic acid molecule comprising a sequence of nucleotides encoding or complementary to a sequence encoding an expression product or a derivative, homolog, analog or mimetic thereof wherein said nucleic acid molecule is expressed in larger amounts in hypothalamus or muscle tissue of fed animals compared to fasted animals.

The expression product may be a protein or mRNA or may be an exon or intron spliced, for example, from an RNA construct.

The terms “lean” and “obese” are used in their most general sense but should be considered relative to the standard criteria for determining obesity. Generally, for human subjects, the definition of obesity is BMI>30 (Risk Factor Prevalence Study Management Committee. Risk Factor Prevalence Study: Survey No. 3 1989. Canberra: National Heart Foundation of Australia and Australian Institute of Health, 1990; Waters and Bennett, Risk Factors for cardiovascular disease: A summary of Australian data. Canberra: Australian Institute of Health and Welfare, 1995).

Conveniently, an animal model may be employed to study the effects of obese and lean animals. In particular, the present invention is exemplified using the Psammomys obesus (the Israeli sand rat) animal model of dietary-induced obesity and NIDDM. In its natural desert habitat, an active lifestyle and saltbush diet ensure that they remair lean and normoglycemic (Shafrir and Gutman, J Basic Clin Physiol Pharm 4: 83-99, 1993). However, in a laboratory setting on a diet of ad libitum chow (on which many other animal species remain healthy), a range of pathophysiological responses are seen (Barnett et a.l, Diabetologia 37: 671-676, 1994a; Barnett et al., Int. J. Obesity 18: 789-794, 1994b, Barnett et al., Diabete Nutr Metab 8: 42-47, 1995). By the age of 16 weeks, more than half of the animals become obese and approximately one third develop NIDDM. Only hyperphagic animals go on to develop hyperglycemia, highlighting the importance of excessive energy intake in the pathophysiology of obesity and NIDDM in Psammomys obesus (Collier et al., Ann New York Acad Sci 827: 50-63, 1997a; Walder et al., Obesity Res 5: 193-200, 1997a). Other phenotypes found include hyperinsulinemia, dyslipidemia and impaired glucose tolerance (Collier et al., [1997a; supra]; Collier et al., Exp Clin Endocrinol Diabetes 105: 36-37, 1997b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al., [1994a; supra]). It is the heterogeneity of the phenotypic response of Psammomys obesus which make it an ideal model to study the etiology and pathophysiology of obesity and NIDDM.

Psammomys obesus animals are conveniently divided into three groups viz Group A animals which are lean, normoglycemic and normoinsulinemic, Group B animals which are obese, normoglycemic and hyperinuslinemic and Group C animals which are obese, hyperglycemic and hyperinsulinemic.

Another aspect of the present invention provides a nucleic acid molecule comprising a nucleotide sequence encoding or complementary to a sequence encoding an expression product or a derivative, homolog, analog or mimetic thereof wherein said nucleotide sequence is as substantially set forth in SEQ ID NO:1 or SEQ ID NO:2 or SEQ ID NO:3 or SEQ ID NO:4 or SEQ ID NO:5 or a nucleotide sequence having at least about 30% similarity to all or part of SEQ ID NO:1 or SEQ ID NO:2 or SEQ ID NO:3 or SEQ ID NO:4 or SEQ ID NO:5 and/or is capable of hybridizing to one or more of SEQ ID NO: 1 or SEQ ID NO:2 or SEQ ID NO:3 or SEQ ID NO:4 or SEQ ID NO:5 or their complementary forms under low stringency conditions at 42° C. and wherein said nucleic acid molecule is expressed in a larger amount in hypothalamus or muscle tissue of obese animals compared to lean animals and/or in fed animals compared to fasted animals.

Reference herein to similarity is generally at a level of comparison of at least 15 consecutive or substantially consecutive nucleotides or at least 5 consecutive or substantially consecutive amino acid residues. Preferred percentage similarities have at least about 40%, at least about 50%, at least about 60%, at least about 70%, at least about 80% and at least about 90% or above.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974. Formamide is optional in these hybridization conditions.

Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The nucleotide sequence or amino acid sequence of the present invention may correspond to exactly the same sequence of the naturally occurring gene (or corresponding cDNA) or protein or may carry one or more nucleotide or amino acid substitutions, additions and/or deletions. The nucleotide sequences set forth in SEQ ID NO: 1, SEQ ID NO:2 or SEQ ID NO:3 or SEQ ID NO:4 or SEQ ID NO:5 correspond to the genes referred to herein as AGT-106, AGT-113, AGT-201, AGT-202 and AGT-203, respectively. The corresponding proteins are AGT-106, AGT-113, AGT-201, AGT-202 and AGT-203, respectively. Reference herein to AGT-106, AGT-113, AGT-201, AGT-202 and AGT-203 includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to AGT-106, AGT-113, AGT-201, AGT-202 and AGT-203.

The expression pattern of AGT-106, AGT-113, AGT-201, AGT-202 and AGT-203 has been determined, inter alia, to indicate an involvement in the regulation of one or more obesity, diabetes and/or energy metabolism. In addition to the differential expression of AGT-106, AGT-113, AGT-201, AGT-202 and AGT-203 in the hypothalamus or muscle tissue of lean versus obese animals and fed versus fasted animals, these genes may also be expressed in other tissues including but in no way limited to muscle and liver. The subject nucleic acid molecules are preferably a sequence of deoxyribonucleic acids such as a cDNA sequence or a genomic sequence. A genomic sequence may also comprise exons and introns. A genomic sequence may also include a promoter region or other regulatory regions. The present invention extends, however, to mRNA, introns and exons which may also be involved in genetic networking, whether or not they are translated into proteins.

A homolog is considered to be a AGT-106, AGT-113, AGT-201, AGT-202 or AGT-203 gene from another animal species. The AGT-106, AGT-113, AGT-201, AGT-202 or AGT-203 gene is exemplified herein from Psammomys obesus hypothalamus. The invention extends, however, to the homologous gene, as determined by nucleotide sequence and/or function, from humans, primates, livestock animals (e.g. cows, sheep, pigs, horses, donkeys), laboratory test animals (e.g. mice, guinea pigs, hamsters, rabbits), companion animals (e.g. cats, dogs) and captured wild animals (e.g. rodents, foxes, deer, kangaroos).

The nucleic acid of the present invention and in particular AGT-106, AGT-113, AGT-201, AGT-202 and AGT-203 and its derivatives and homologs may be in isolated or purified form and/or may be ligated to a vector such as an expression vector. Expression may be in a eukaryotic cell line (e.g. mammalian, insect or yeast cells) or in microbial cells (e.g. E. coli) or both.

The derivatives of the nucleic acid molecule of the present invention include oligonucleotides, PCR primers, antisense molecules, molecules suitable for use in co-suppression and fusion nucleic acid molecules. Ribozymes and DNA enzymes are also contemplated by the present invention directed to AGT-106, AGT-113, AGT-201, AGT-202 and AGT-203 or its mRNA. Derivatives and homologs of AGT-106, AGT-113, AGT-201, AGT-202 and AGT-203 are conveniently encompassed by those nucleotide sequences capable of hybridizing to SEQ ID NO: 1, SEQ ID NO:2 or SEQ ID NO:3 or SEQ ID NO:4 or SEQ ID NO:5 under low stringency conditions at 42° C.

The present invention extends to expression products of AGT-106, AGT-113, AGT-201, AGT-202 and AGT-203. The preferred expression products are proteins or mutants, derivatives, homologs or analogs thereof.

Derivatives include fragments, parts, portions, mutants, variants and mimetics from natural, synthetic or recombinant sources including fusion proteins. Parts or fragments include, for example, active regions of AGT-106, AGT-113, AGT-202 or AGT-203. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of AGT-106, AGT-113, AGT-202 or AGT-203 should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Another aspect of the present invention provides an isolated protein or a derivative, homolog, analog or mimetic thereof which is produced in larger amounts in hyperthalamus tissue in obese animals compared to lean animals.

In a more preferred aspect of the present invention, there is provided an isolated protein or a derivative, homolog, analog or mimetic thereof wherein said protein comprises an amino acid sequence substantially encoded by a nucleotide sequence as set forth in SEQ ID NO: 1, SEQ ID NO:2, SEQ ID NO:3, SEQ ID NO:4 or SEQ ID NO:5 or an amino acid sequence having at least 30% similarity to all or part thereof and wherein said protein is produced in a larger amount in hypothalamus or muscle tissue of obese animals compared to lean animals.

A further aspect of the present invention is directed to an isolated protein or a derivative, homolog, analog or mimetic thereof wherein said protein is encoded by a nucleotide sequence substantially as set forth in SEQ ID NO: 1, SEQ ID NO:2, SEQ ID NO:3, SEQ ID NO:4 or SEQ ID NO:5 or a nucleotide sequence having at least 30% similarity to all or part of SEQ ID NO:1, SEQ ID NO:2, SEQ ID NO:3, SEQ ID NO:4 or SEQ ID NO:5 and/or is capable of hybridizing to SEQ ID NO: 1, SEQ ID NO:2, SEQ ID NO:3, SEQ ID NO:4 or SEQ ID NO:5 or their complementary forms under low stringency conditions at 42° C.

Reference herein to AGT-106, AGT-113, AGT-201, AGT-202 and AGT-203 includes reference to isolated or purified naturally occurring AGT-106, AGT-113, AGT-201, AGT-202 and AGT-203 protein molecules as well as any derivatives, homologs, analogs and mimetics thereof. Derivatives include parts, fragments and portions of AGT-106, AGT-113, AGT-201, AGT-202 and AGT-203 as well as single and multiple amino acid substitutions, deletions and/or additions to AGT-106, AGT-113, AGT-201, AGT-202 and AGT-203. A derivative of AGT-106, AGT-113, AGT-201, AGT-202 and AGT-203 is conveniently encompassed by molecules encoded by a nucleotide sequence capable of hybridizing to SEQ ID NO: 1, SEQ ID NO:2, SEQ ID NO:3, SEQ ID NO:4 or SEQ ID NO:5 under low stringency conditions at 42° C.

Other derivatives of AGT-106, AGT-113, AGT-201, AGT-202 and AGT-203 include chemical analogs. Analogs of AGT-106, AGT-113, AGT-201, AGT-202 and AGT-203 contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH4.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A list of unnatural amino acid, contemplated herein is shown in Table 3.

TABLE 3

| | | Non-conventional | |
|---|---|---|---|
| Non-conventional amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

All such modifications may also be useful in stabilizing the AGT-106, AGT-113, AGT-201, AGT-202 and AGT-203 molecule for use in in vivo administration protocols or for diagnostic purposes.

The nucleic acid molecule of the present invention is preferably in isolated form or ligated to a vector, such as an expression vector. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the nucleotide sequence corresponding to AGT-106 is a cDNA sequence comprising a sequence of nucleotides as set forth in SEQ ID NO: 1 or a derivative, homolog or analog thereof including a nucleotide sequence having similarity to SEQ ID NO:1.

In another particularly preferred embodiment, the nucleotide sequence corresponding to AGT-113 is a EDNA sequence comprising a sequence of nucleotides as set forth in SEQ ID NO:2 or a derivative, homolog or analog thereof including a nucleotide sequence having similarity to SEQ ID NO:3.

In still another particularly preferred embodiment, the nucleotide sequence corresponding to AGT-201 is a cDNA sequence comprising a sequence of nucleotides as set forth in SEQ ID NO:3 or a derivative, homolog or analog thereof including a nucleotide sequence having similarity to SEQ ID NO:3.

In a further particularly preferred embodiment, the nucleotide sequence corresponding to AGT-202 is a cDNA sequence comprising a sequence of nucleotides as set forth in SEQ ID NO:4 or a derivative, homolog or analog thereof including a nucleotide sequence having similarity to SEQ ID NO:4.

In still a further particularly preferred embodiment, the nucleotide sequence corresponding to AGT-203 is a cDNA sequence comprising a sequence of nucleotides as set forth in SEQ ID NO:5 or a derivative, homolog or analog thereof including a nucleotide sequence having similarity to SEQ ID NO:5.

The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of sphingosine kinase which forms are encompassed by the present invention.

The present invention extends to the expression product of the nucleic acid molecules as hereinbefore defined. The expression product is preferably a protein but extends to mRNA, RNA, introns and exons.

Preferably, the expression products are AGT-106, AGT-113, AGT-201, AGT-202 and AGT-203 encoded by SEQ ID NO:1, SEQ ID NO:2, SEQ ID NO:3, SEQ ID NO:4 or SEQ ID NO:5, respectively or are derivatives, analogs, homologs, chemical equivalents or mimetics thereof.

Another aspect of the present invention is directed to an isolated protein selected from the list consisting of:—
(i) a protein encoded by a nucleic acid molecule which molecule is differentially expressed in hypothalamus or muscle tissue of obese animals compared to lean animals or a derivative, homolog, analog, chemical equivalent or mimetic thereof;
(ii) a protein encoded by a nucleic acid molecule which molecule is differentially expressed in liver tissue of fed animals compared to fasted animals or a derivative, homolog, analog, chemical equivalent or mimetic thereof;
(iii) a protein encoded by a nucleotide sequence substantially as set forth in SEQ ID NO: 1 or a derivative, homolog or analog thereof or a sequence encoding an amino acid sequence having at least about 30% similarity to this sequence or a derivative, homolog, analog, chemical equivalent or mimetic of said protein;
(iv) a protein encoded by a nucleotide sequence substantially as set forth in SEQ ID NO:2 or a derivative, homolog or analog thereof or a sequence encoding an amino acid sequence having at least about 30% similarity to this sequence or a derivative, homolog, analog, chemical equivalent or mimetic of said protein;
(v) a protein encoded by a nucleotide sequence substantially as set forth in SEQ ID NO:3 or a derivative, homolog or analog thereof or a sequence encoding an amino acid sequence having at least about 30% similarity to this sequence or a derivative, homolog, analog, chemical equivalent or mimetic of said protein;
(vi) a protein encoded by a nucleotide sequence substantially as set forth in SEQ ID NO:4 or a derivative, homolog or analog thereof or a sequence encoding an amino acid sequence having at least about 30% similarity to this sequence or a derivative, homolog, analog, chemical equivalent or mimetic of said protein;
(vii) a protein encoded by a nucleotide sequence substantially as set forth in SEQ ID NO:5 or a derivative, homolog or analog thereof or a sequence encoding an amino acid sequence having at least about 30% similarity to this sequence or a derivative, homolog, analog, chemical equivalent or mimetic of said protein;
(viii) a protein encoded by a nucleic acid molecule capable of hybridizing to the nucleotide sequence as set forth in SEQ ID NO:1 or a derivative, homolog or analog thereof under low stringency conditions;
(ix) a protein encoded by a nucleic acid molecule capable of hybridizing to the nucleotide sequence as set forth in SEQ ID NO:2 or a derivative, homolog or analog thereof under low stringency conditions;
(x) a protein encoded by a nucleic acid molecule capable of hybridizing to the nucleotide sequence as set forth in SEQ ID NO:3 or a derivative, homolog or analog thereof under low stringency conditions;
(xi) a protein encoded by a nucleic acid molecule capable of hybridizing to the nucleotide sequence as set forth in SEQ ID NO:4 or a derivative, homolog or analog thereof under low stringency conditions;
(xii) a protein encoded by a nucleic acid molecule capable of hybridizing to the nucleotide sequence as set forth in SEQ ID NO:5 or a derivative, homolog or analog thereof under low stringency conditions;
(xiii) a protein as defined in any one of paragraphs (i) to (xii) in a homodimeric form; and
(vi) a protein as defined in any one of paragraphs (i) to (xii) in a heterodimeric form.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

Without limiting the theory or mode of action of the present invention, the expression of AGT-106, AGT-113, AGT-201, AGT-202 and/or AGT-203 is thought to relate to body weight and circulating triglycerides. Modulation of these genes expression is thought, inter alia, to regulate energy balance via effects on energy intake and also effects on carbohydrate/fat metabolism. The energy intake effects are likely to be mediated via the central nervous system but peripheral effects on the metabolism of both carbohydrate and fat are possible. The expression of these genes may also be regulated by fasting and feeding, accordingly, regulating the expression and/or activity of these genes or their expression products could provide a mechanism for regulating both body weight and energy metabolism, including carbohydrate and fat metabolism.

The identification of AGT-106, AGT-113, AGT-201, AGT-202 and AGT-203 permits the generation of a range of therapeutic molecules capable of modulating expression of AGT-106, AGT-113, AGT-201, AGT-202 and AGT-203 or modulating the activity of AGT-106, AGT-113, AGT-201, AGT-202 and AGT-203. Modulators contemplated by the present invention includes agonists and antagonists of AGT-106, AGT-113, AGT-201, AGT-202 and AGT-203 expression. Antagonists of AGT-106, AGT-113, AGT-201, AGT-202 and AGT-203 expression include antisense molecules, ribozymes and co-suppression molecules. Agonists include molecules which increase promoter activity or which interfere with negative regulatory mechanisms. Antagonists of AGT-106, AGT-113, AGT-201, AGT-202 and AGT-203 include antibodies and inhibitor peptide fragments. All such molecules may first need to be modified to enable such molecules to penetrate cell membranes. Alternatively, viral agents may be employed to introduce genetic elements to modulate expression of AGT-106, AGT-113, AGT-201, AGT-202 and AGT-203. In so far as AGT-106, AGT-113, AGT-201, AGT-202 and AGT-203 acts in association with other genes such as the ob gene which encodes leptin, the therapeutic molecules may target the AGT-106, AGT-113, AGT-201, AGT-202 and AGT-203 and ob genes or their translation products.

The present invention contemplates, therefore, a method for modulating expression of one or more of AGT-106, AGT-113, AGT-201, AGT-202 and AGT-203 in a mammal, said method comprising contacting the AGT-106, AGT-113, AGT-201, AGT-202 and AGT-203 gene with an effective amount of a modulator of AGT-106, AGT-113, AGT-201, AGT-202 and/or AGT-203 expression for a time and under conditions sufficient to up-regulate or down-regulate or otherwise modulate expression of AGT-106, AGT-113, AGT-201, AGT-202 and AGT-203. For example, a nucleic acid molecule encoding AGT-106, AGT-113, AGT-201, AGT-202 and AGT-203 or a derivative or homolog thereof may be introduced into a cell to enhance the ability of that cell to produce AGT-106, AGT-113, AGT-201, AGT-202 and AGT-203, conversely, AGT-106, AGT-113, AGT-201, AGT-202 and AGT-203 antisense sequences such as oligonucleotides may be introduced to decrease the availability of AGT-106, AGT-113, AGT-201, AGT-202 and AGT-203 molecules.

Another aspect of the present invention contemplates a method of modulating activity of AGT-106, AGT-113, AGT-201, AGT-202 and AGT-203 in a mammal, said method comprising administering to said mammal a modulating effective amount of a molecule for a time and under conditions sufficient to increase or decrease AGT-106, AGT-113, AGT-201, AGT-202 and AGT-203 activity. The molecule may be a proteinaceous molecule or a chemical entity and may also be a derivative of AGT-106, AGT-113, AGT-201, AGT-202 and AGT-203 or its ligand.

Modulating levels of AGT-106, AGT-113, AGT-201, AGT-202 and AGT-203 expression is important in the treatment of a range of conditions such as obesity, anorexia, energy imbalance, diabetes, metabolic syndrome, dyslipidemia, hypertension, insulin resistance and muscle development conditions. It may also be useful in the agricultural industry to assist in the generation of leaner animals, or where required, more obese animals. Accordingly, the mammal contemplated by the present invention includes but is not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates therapeutic and prophylactic uses of AGT-106, AGT-113, AGT-201, AGT-202 and/or AGT-203 amino acid and nucleic acid molecules in addition to AGT-106, AGT-113, AGT-201, AGT-202 and/or AGT-203 agonistic and antagonistic agents.

The present invention contemplates, therefore, a method of modulating expression of AGT-106, AGT-113, AGT-201, AGT-202 and/or AGT-203 in a mammal, said method comprising contacting the AGT-106, AGT-113, AGT-201, AGT-202 and/or AGT-203 genes with an effective amount of an agent for a time and under conditions sufficient to up-regulate, down-regulate or otherwise module expression of AGT-106, AGT-113, AGT-201, AGT-202 and/or AGT-203. For example, antisense sequences such as oligonucleotides may be utilized.

Conversely, nucleic acid molecules encoding AGT-106, AGT-113, AGT-201, AGT-202 and/or AGT-203 or derivatives thereof may be introduced to up-regulate one or more specific functional activities.

Another aspect of the present invention contemplates a method of modulating activity of AGT-106, AGT-113, AGT-201, AGT-202 and/or AGT-203 in a subject, said method comprising administering to said subject a modulating effective amount of an agent for a time and under conditions sufficient to increase or decrease AGT-106, AGT-113, AGT-201, AGT-202 and/or AGT-203 activity.

Modulation of said activity by the administration of an agent to a mammal can be achieved by one of several techniques, including but in no way limited to introducing into said mammal a proteinaceous or non-proteinaceous molecule which:
(i) modulates expression of AGT-106, AGT-113, AGT-201, AGT-202 and/or AGT-203;
(ii) functions as an antagonist of AGT-106, AGT-113, AGT-201, AGT-202 and/or AGT-203;
(iii) functions as an agonist of AGT-106, AGT-113, AGT-201, AGT-202 and/or AGT-203.

The proteinaceous molecule may be derived from natural or recombinant sources including fusion proteins or following, for example, natural product screening. Said non-proteinaceous molecule may be, for example, a nucleic acid molecule or may be derived from natural sources, such as for example natural product screening or may be chemically synthesized. The present invention contemplates chemical analogs of AGT-106, AGT-113, AGT-201, AGT-202 and/or AGT-203 or small molecules capable of acting as agonists or antagonists. Chemical agonists may not necessarily be derived from AGT-106, AGT-113, AGT-201, AGT-202 and/or AGT-203 but may share certain conformational similarities. Alternatively, chemical agonists may be specifically designed to mimic certain physiochemical properties. Antagonists may be any compound capable of blocking, inhibiting or otherwise preventing AGT-106, AGT-113, AGT-201, AGT-202 and/or AGT-203 from carrying out their normal biological functions. Antagonists include monoclonal antibodies and antisense nucleic acids which prevent transcription or translation of AGT-106, AGT-113, AGT-201, AGT-202 and/or AGT-203 genes or mRNA in mammalian cells. Modulation of expression may also be achieved utilizing antigens, RNA, ribosomes, DNAzymes, RNA aptamers or antibodies.

The proteinaceous or non-proteinaceous molecule may act either directly or indirectly to modulate the expression of AGT-106, AGT-113, AGT-201, AGT-202 and/or AGT-203 or the activity of AGT-106, AGT-113, AGT-201, AGT-202 and/or AGT-203. Said molecule acts directly if it associates with AGT-106, AGT-113, AGT-201, AGT-202 and/or AGT-203 or AGT-106, AGT-113, AGT-201, AGT-202 and/or AGT-203 to modulate expression or activity. Said molecule acts indirectly if it associates with a molecule other than AGT-106, AGT-113, AGT-201, AGT-202 and/or AGT-203 or AGT-106, AGT-113, AGT-201, AGT-202 and/or AGT-203 which other molecule either directly or indirectly modulates the expression or activity of AGT-106, AGT-113, AGT-201, AGT-202 and/or AGT-203 or AGT-106, AGT-113, AGT-201, AGT-202 and/or AGT-203. Accordingly, the method of the present invention encompasses the regulation of AGT-106, AGT-113, AGT-201, AGT-202 and/or AGT-203 or AGT-106, AGT-113, AGT-201, AGT-202 and/or AGT-203 expression or activity via the induction of a cascade of regulatory steps.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful but in no way limited to use in a therapeutic or prophylactic treatment of obesity, anorexia, diabetes or energy imbalance.

Accordingly, another aspect of the present invention relates to a method of treating a mammal suffering from a condition characterized by one or more symptoms of obesity, anorexia, diabetes and/or energy imbalance, said method comprising administering to said mammal an effective amount of an agent for a time and under conditions sufficient to modulate the expression of AGT-106, AGT-113, AGT-201, AGT-202 and/or AGT-203 or sufficient to modulate the activity of AGT-106, AGT-113, AGT-201, AGT-202 and/or AGT-203.

In another aspect, the present invention relates to a method of treating a mammal suffering from a disease condition characterized by one or more symptoms of obesity, anorexia, diabetes or energy imbalance, said method comprising administering to said mammal an effective amount of AGT-106, AGT-113, AGT-201, AGT-202 and/or AGT-203 or AGT-106, AGT-113, AGT-201, AGT-202 and/or AGT-203.

An “effective amount” means an amount necessary at least partly to attain the desired immune response, or to delay the onset or inhibit progression or halt altogether, the onset or progression of a particular condition of the individual to be treated, the taxonomic group of the individual to be treated, the degree of protection desired, the formulation of the vaccine, the assessment of the medical situation, and other relevant factors. It is expected that the amount will fall in a relatively broad range that can be determined through routine trials.

In accordance with these methods, AGT-106, AGT-113, AGT-201, AGT-202 and/or AGT-203 or AGT-106, AGT-113, AGT-201, AGT-202 and/or AGT-203 or agents capable of modulating the expression or activity of said molecules may be co-administered with one or more other compounds or other molecules. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect, the present invention relates to the use of an agent capable of modulating the expression of AGT-106, AGT-113, AGT-201, AGT-202 and/or AGT-203 or a derivative, homolog or analog thereof in the manufacture of a medicament for the treatment of a condition characterized by obesity, anorexia, diabetes and/or energy imbalance.

In still yet another aspect, the present invention relates to the use of an agent capable of modulating the activity of AGT-106, AGT-113, AGT-201, AGT-202and/or AGT-203 or a derivative, homolog, analog, chemical equivalent or mimetic thereof in the manufacture of a medicament for the treatment of a condition characterized by obesity, anorexia, diabetes and/or energy imbalance.

A further aspect of the present invention relates to the use of AGT-106, AGT-113, AGT-201, AGT-202 and/or AGT-203 or derivative, homolog or analog thereof or AGT-106, AGT-113, AGT-201, AGT-202 and/or AGT-203 or derivative, homolog, analog, chemical equivalent or mimetic thereof in the manufacture of a medicament for the treatment of a condition characterized by obesity, anorexia, diabetes, impaired muscle development and/or energy imbalance.

Still yet another aspect of the present invention relates to agents for use in modulating the expression of AGT-106, AGT-113, AGT-201, AGT-202 and/or AGT-203 or a derivative, homolog or analog thereof.

A further aspect relates to agents for use in modulating AGT-106, AGT-113, AGT-201, AGT-202 and/or AGT-203 activity or a derivative, homolog, analog, chemical equivalent or mimetic thereof.

Still another aspect of the present invention relates to AGT-106, AGT-113, AGT-201, AGT-202 and/or AGT-203 or derivative, homolog or analog thereof or AGT-106, AGT-113, AGT-201, AGT-202 and/or AGT-203 or derivative, homolog, analog, chemical equivalent or mimetic thereof for use in treating a condition characterized by one or more symptoms of obesity, anorexia, diabetes, impaired muscle development and/or energy imbalance.

In a related aspect of the present invention, the mammal undergoing treatment may be a human or an animal in need of therapeutic or prophylactic treatment.

Accordingly, the present invention contemplates in one embodiment a composition comprising a modulator of AGT-106, AGT-113, AGT-201, AGT-202 and AGT-203 expression or AGT-106, AGT-113, AGT-201, AGT-202 and AGT-203 activity and one or more pharmaceutically acceptable carriers and/or diluents. In another embodiment, the composition comprises AGT-106, AGT-113, AGT-201, AGT-202 and AGT-203 or a derivative, homolog, analog or mimetic thereof and one or more pharmaceutically acceptable carriers and/or diluents. The compositions may also comprise leptin or modulations of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fingi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When AGT-106, AGT-113, AGT-201, AGT-202 and AGT-203 and AGT-106, AGT-113, AGT-201, AGT-202 and AGT-203 including AGT-106, AGT-113, AGT-201, AGT-202 and AGT-203 itself are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of AGT-106, AGT-113, AGT-201, AGT-202 and AGT-203 will range from 0.01 ng/kg/body weight to above 10,000 mg/kg/body weight. Alternative amounts range from 0.1 ng/kg/body weight is above 1000 mg/kg/body weight. AGT-106, AGT-113, AGT-201, AGT-202 and AGT-203 may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating AGT-106, AGT-113, AGT-201, AGT-202 and AGT-203 expression or AGT-106, AGT-113, AGT-201, AGT-202 and AGT-203 activity. The vector may, for example, be a viral vector.

Still another aspect of the present invention is directed to antibodies to AGT-106, AGT-113, AGT-201, AGT-202 and AGT-203 and their derivatives and homologs. Such antibodies may be monoclonal or polyclonal and may be selected from naturally occurring antibodies to AGT-106, AGT-113, AGT-201, AGT-202 and AGT-203 or may be specifically raised to AGT-106, AGT-113, AGT-201, AGT-202 and AGT-203 or derivatives or homologs thereof. In the case of the latter, AGT-106, AGT-113, AGT-201, AGT-202 and AGT-203 or their derivatives or homologs may first need to be associated with a carrier molecule. The antibodies and/or recombinant AGT-106, AGT-113, AGT-201, AGT-202 and AGT-203 or their derivatives of the present invention are particularly useful as therapeutic or diagnostic agents.

For example, AGT-106, AGT-113, AGT-201, AGT-202 and AGT-203 and their derivatives can be used to screen for naturally occurring antibodies to AGT-106, AGT-113, AGT-201, AGT-202 and AGT-203 which may occur in certain autoimmune diseases or where cell death is occurring. These may occur, for example in some autoimmune diseases. Alternatively, specific antibodies can be used to screen for AGT-106, AGT-113, AGT-201, AGT-202 and AGT-203. Techniques for such assays are well known in the art and include, for example, sandwich assays and ELISA.

Antibodies to AGT-106, AGT-113, AGT-201, AGT-202 and AGT-203 of the present invention may be monoclonal or polyclonal and may be selected from naturally occurring antibodies to the AGT-106, AGT-113, AGT-201, AGT-202 and AGT-203 or may be specifically raised to the AGT-106, AGT-113, AGT-201, AGT-202 and AGT-203 or their derivatives. In the case of the latter, the AGT-106, AGT-113, AGT-201, AGT-202 and AGT-203 protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying AGT-106, AGT-113, AGT-201, AGT-202 and AGT-203.

For example, specific antibodies can be used to screen for AGT-106, AGT-113, AGT-201, AGT-202 and AGT-203 proteins. The latter would be important, for example, as a means for screening for levels of AGT-106, AGT-113, AGT-201, AGT-202 and AGT-203 in a cell extract or other biological fluid or purifying AGT-106, AGT-113, AGT-201, AGT-202 and AGT-203 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of AGT-106, AGT-113, AGT-201, AGT-202 and AGT-203.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of AGT-106, AGT-113, AGT-201, AGT-202 and AGT-203, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffman, Basic Facts about Hybridomas, in Compendium of Immunology Vol. II, ed. by Schwartz, 1981; Kohler and Milstein, Nature 256: 495499, 1975; Kohler and Milstein, European Journal of Immunology 6: 511-519,1976).

Another aspect of the present invention contemplates a method for detecting AGT-106, AGT-113, AGT-201, AGT-202 and AGT-203 or a derivative or homolog thereof in a biological sample from a subject, said method comprising contacting said biological sample with an antibody specific for AGT-106, AGT-113, AGT-201, AGT-202 and AGT-203 or their antigenic derivatives or homologs for a time and under conditions sufficient for a complex to form, and then detecting said complex.

The presence of the complex is indicative of the presence of AGT-106, AGT-113, AGT-201, AGT-202 and AGT-203. This assay may be quantitated or semi-quantitated to determine a propensity to develop obesity or other conditions or to monitor a therapeutic regimum.

The presence of AGT-106, AGT-113, AGT-201, AGT-202 and AGT-203 may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-AGT-106, AGT-113, AGT-201, AGT-202 and AGT-203 complex, a second antibody specific to the AGT-106, AGT-113, AGT-201, AGT-202 and AGT-203, labelled with a reporter molecule capable of producing a detectable signal, is then added and incubated, allowing time sufficient for the formation of another complex of antibody-AGT-106, AGT-113, AGT-201, AGT-202 and AGT-203-labelled antibody. Any unreacted material is washed away, and the presence of AGT-106, AGT-113, AGT-201, AGT-202 and AGT-203 is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain AGT-106, AGT-113, AGT-201, AGT-202 and AGT-203 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of any subunit present in the antibody. Following the incubation period, the antibody subunit solid phase is washed and dried and incubated with a second antibody specific for a portion of AGT-106, AGT-113, AGT-201, AGT-202 and AGT-203. The second antibody is linked to a reporter molecule which is used to indicate the binding of the second antibody to AGT-106, AGT-113, AGT-201, AGT-202 and AGT-203.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. A “reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect AGT-106, AGT-113, AGT-201, AGT-202 and AGT-203 or their derivatives.

The assays of the present invention may also extend to measuring AGT-106, AGT-113, AGT-201, AGT-202 and AGT-203 or AGT-106, AGT-113, AGT-201, AGT-202 and AGT-203 in association with ob or leptin.

The present invention is further described by the following non-limiting Examples.

### EXAMPLE 1

### Psammomys obesus Colony

A Psammomys obesus colony is maintained at Deakin University, Waurn Ponds, Geelong, Victoria, Australia with the breeding pairs fed ad libitum a diet of lucerne and standard laboratory chow. Animals are weaned at four weeks of age and sustained on a diet of standard laboratory chow from which 12% of energy was derived from fat, 63% from carbohydrate and 25% from protein (Barastoc, Pakenham, Australia). Animals are housed in a humidity and temperature controlled room (22±1° C.) with a 12-12-hour light-dark cycle.

Group A animals are lean, normoglycemic and normoinsulinemic, group B animals are obese, normoglycemic and hyperinsulinemic, and group C animals are obese, hyperglycemic and hyperinsulinemia.

### EXAMPLE 2

### Experimental Animals

At 18 weeks of age the animals were weighed and blood collected from the tail vein in the fed state. The animals were sacrificed and the tissues immediately removed, weighed, frozen in liquid nitrogen and stored at −80° C. Percent body fat was estimated from the combined weight of mesenteric, suprascapular, perirenal, epididymal and intramuscular (from between the heads of gastrocnemius) fat pads expressed as a percentage of total body weight. Animals were classified into groups A, B or C based on their blood glucose and insulin concentrations. The cutoff values for hyperglycemia and hyperinsulinemia were 8 mmol/L and 150 μU/ml respectively. The fasted animals were weighed and bled in the fed state then fasted for 24 hours, before being weighed and bled again before sacrifice.

### EXAMPLE 3

### Analytical Methods

Whole blood glucose was measured immediately using an enzymatic glucose analyser (Model 27, Yellow Springs Instruments, Ohio). Plasma insulin concentrations were determined using a double antibody solid phase radioimmunoassay (Phadeseph, Kabi Pharmacia Diagnostics, Sweden).

### EXAMPLE 4

### RNA Extraction and Reverse Transcription

RNA was extracted from tissues using TriZol (Life Technologies, Rockville, Md.) according to manufacturer's recommendations for each tissue type. RNA was quantitated by spectrophotometry at 260 nm (Beckman Instruments, Fullerton, Calif.) and 2 μg electrophoresed through a 1% w/v glyoxal agarose gel (Ambion, Austin, Tex.) to check the integrity. The RNA was then reverse transcribed using AMV reverse transcriptase with oligo(dT) primers (Promega, Madison, Wis.).

### EXAMPLE 5

### Statistical Analysis

All data are expressed as mean ±S.E.M. A one-way analysis of variance in combination with post hoc least significant difference or Games-Howell test were used to compare means between groups, and t-tests were used where appropriate. A 2-tailed Pearson correlation was performed to analyse relationships between gene expression and phenotypes. Blood glucose and plasma insulin concentrations were log transformed prior to analysis to approximate a normal distribution. Differences were considered significant at P<0.05.

### EXAMPLE 6

### Differential Display PCR

RNA was extracted from tissues using TriZol (Life Technologies, Rockville, Md.) and DNase-treated (Life Technologies), phenol:chloroform (4:1) extracted and ethanol precipitated. RNA was quantitated by spectrophotometry at 260 nm (Beckman Instruments, Fullerton, Calif.) and 2 μg electrophoresed through a 1% w/v glyoxal agarose gel (Ambion, Austin, Tex.) to check the integrity. The RNA was then reverse transcribed using Superscript II reverse transcriptase (Life Technologies) with anchored oligo(dT) primers. Differential display PCR was performed on hypothalamic cDNA samples from obese and lean Psammomys obesus in the fed and fasted state using an RNAimage mRNA Differential Display System (GenHunter Corporation, Nashville, Tenn.). The PCR products were separated on a 6% w/v polyacrylamide gel, and differentially expressed PCR fragments were visualized by exposing the dried gel to x-ray film. Candidate bands were excised from the gel and reamplified by PCR using the appropriate primer combination. Sequencing reactions were carried out using ABI PRISM Big-Dye terminator cycle sequencing ready reaction kits and analyzed on an ABI 373A DNA sequencer. Gene database searches were performed at the National Centre for Biotechnology Information using the BLAST network service.

### EXAMPLE 7

### Macroarray

RNA was extracted from tissues using TriZol (Life Technologies, Rockville, Md.) and DNase-treated (Life Technologies), phenol:chloroform (4:1) extracted and ethanol precipitated. RNA was quantitated by spectrophotometry at 260 nm (Beckman Instruments, Fullerton, Calif.) and 2 μg electrophoresed through a 1% w/v glyoxal agarose gel (Ambion, Austin, Tex.) to check the integrity. Pooled RNA samples for each of the study groups were labeled with ³³P d-ATP, and hybridiszd to human GF 201 membrane microarray filters (Research Genetics). The membrane was spotted with a total of 5184 genes, including known genes and expressed sequence tags. The level of binding to each gene was quantitated using a phosphorimager and compared using Pathways software (Research Genetics).

### EXAMPLE 8

### AGT-106

AGT-106 was identified as being differentially expressed using the technique of differential display PCR in the hypothalamus of the Israeli Sand Rat (ISR) and expression of AGT-106 was higher in fed compared to fasted Sand rats.

Primers used were:—

AGT-106:

| | |
|---|---|
| Forward primer: | |
| 5′-CAATCACCGCTTTTAAGATAGTTTGT-3′ | [SEQ ID NO: 12] |
| Reverse primer: | |
| 5′-AGCATTAAAAAGGGCTCGCA-3′ | [SEQ ID NO: 13] |

The partial nucleotide sequence of Psammomys obesus AGT-106 cDNA is as follows:—

| | |
|---|---|
| NTTTGNTGNCCNGCTGTGTGTGTTAGAAGAAAACAG | [SEQ ID NO: 1] |
| AAAAGGAAAGAAAAACAATCACCGCTTTTAAGATAG | |
| TTTGTATCAGCTTAGATTTCATCATGACTGTTTTAC | |
| ATACTGGAATTTATAAATTGTAAGTTATCATTTTCC | |
| AATGCGAGCCCTTTTTAATGCTTTTTAAAACTTGTG | |
| AATAAAATTGATACTCCTT. | |

Blast analysis revealed sequence homology between the AGT-106 cDNA sequence and murine TROY mRNA. TROY is a newly identified member of the Tumour Necrosis Factor Receptor Superfamily (Kojima et al., J. Biol. Chem. 275(27): 20742-20747, 2000). Nucleotide sequence homology of ISR TROY with mouse TROY mRNA is 85%.

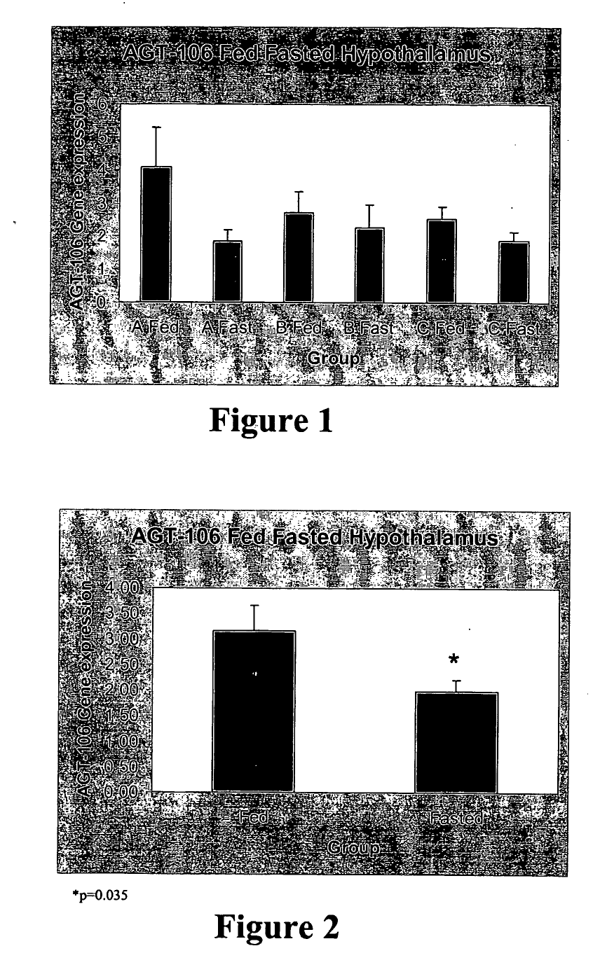

Hypothalamic AGT-106 expression was decreased with fasting in Psammomys obesus (FIGS. 1, 2). The main effect was observed in group A fed and A fasted animals with an approximately 50% reduction in AGT-106 expression with fasting. This dramatic reduction in AGT-106 expression with fasting was not evident in group B and C animals, and the fed animals in both of these obese groups were similar to the fasted group A animals. These results demonstrate that hypothalamic expression of AGT-106 in obese animals remains suppressed even in the fed state, suggesting a dysregulation of this gene in these animals.

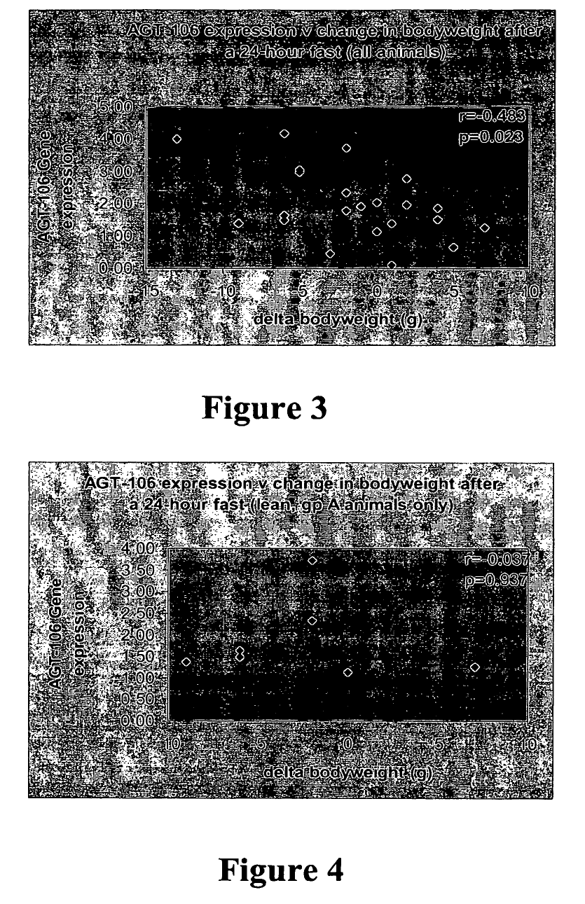

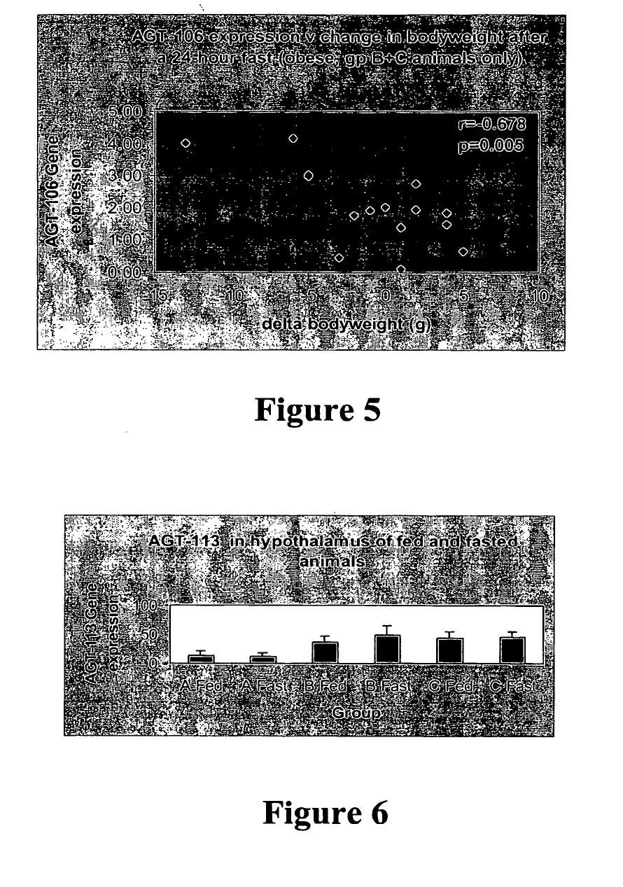

Interestingly, a significant relationship was also demonstrated between the change in bodyweight after the 24-hour fast (delta bodyweight) and AGT-106 gene expression in the hypothalamus (FIG. 3). Although this relationship was seen when all animals were combined, when animals were separated into lean and obese, the association disappeared in the lean animals (FIG. 4), but strengthened in the obese groups (FIG. 5). There was no relationship between hypothalamic AGT-106 expression and circulating glucose or insulin concentrations.

This demonstrates, therefor, a novel role for Troy (AGT-106), a member of the Tumor Necrosin Factor Receptor Superfamily (TNFRSF) in the regulation of energy utilisation and bodyweight in response to fasting in rodents. As AGT-106 is a receptor in the hypothalamus, a key site within the brain for the regulation of bodyweight and energy balance, this regulation may involve downstream transcriptional regulation of genes involved in homeostasis via the NF-κB pathway, or other as yet unidentified pathways. Alternatively, this regulation may involve the action of circulating messengers/molecules feeding back information to the hypothalamus on the state of energy balance within the body.

### EXAMPLE 9

### AGT-113

AGT-113 was discovered using differential display and it appeared to be expressed at higher levels in the hypothalamus of group C (obese, diabetic) animals than group A (lean healthy) animals.

Real time PCR confirmed this (FIG. 6), and showed that group A animals, in both the fed and fasted state, have a much lower expression level of this gene in their hypothalamus than group B (obese, impaired glucose tolerant) animals and group C animals (A fed v C fed, p=0.031; A fast v B fast, p=0.028; A fast v C fast, p=0.023).

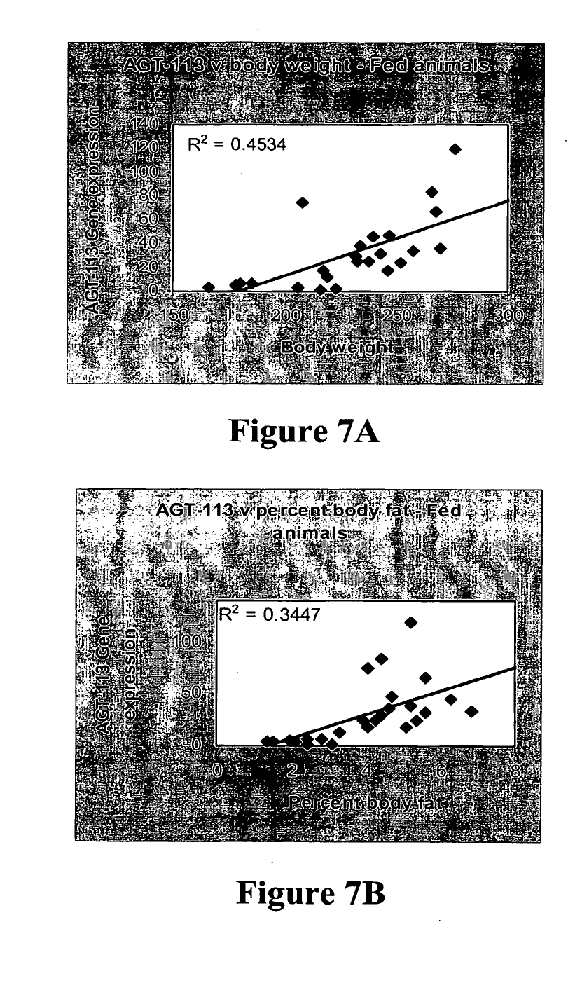

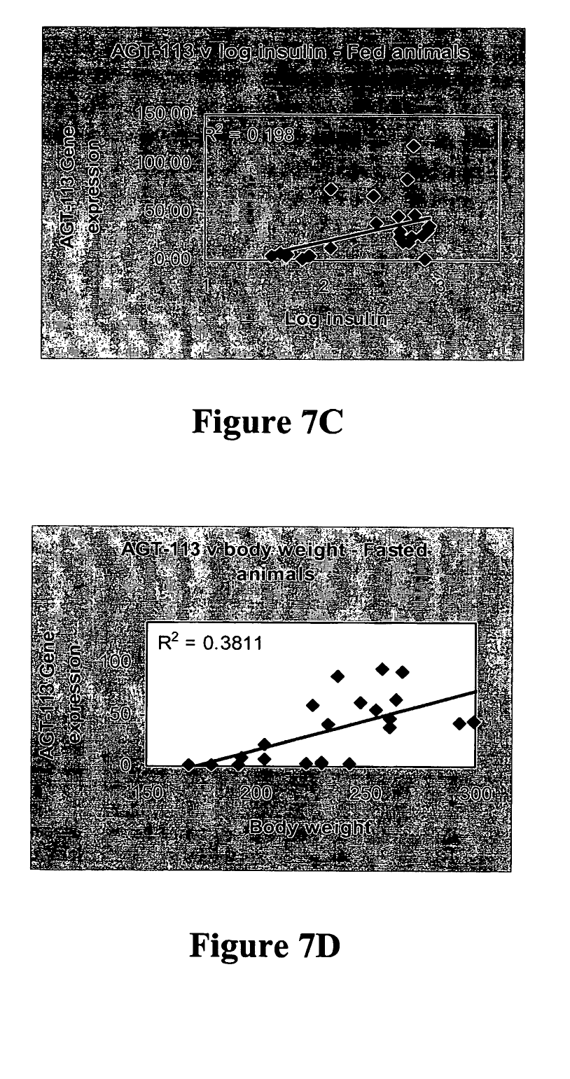

In the fed state, hypothalamic AGT-113 gene expression correlated with body weight (p<0.001, FIG. 7A), percent body fat (p=0.002, FIG. 7B) and plasma insulin levels (p=0.026, FIG. 7C). In the fasted state, hypothalamic AGT-113 gene expression correlated with body weight only (p=0.002, FIG. 7D).

Gene expression in each cDNA sample was quantitated using Taqman PCR technology on an ABI Prism 7700 sequence detector (PE Applied Biosystems, Foster City, Calif.). Cyclophilin was used as an endogenous control to standardise the amount of cDNA added to a reaction. PCR conditions were 50° C. for 2 min, 95° C. for 10 min followed by 40 cycles of 95° C. for 15 sec and 60° C. for 1 min. All samples were assayed in duplicate. For. AGT-201 (Example 10), AGT-203 (Example 11) and cyclophilin, fluorogenic probes which had the reporter dye FAM attached to the 5′ end and the quencher dye TAMRA attached to the 3′ end were used with Taqman Universal PCR Master Mix (PE Applied Biosystems). For AGT-202, AGT-106 (Troy) and AGT-113, no probe was used, and SYBR Green Master Mix (PE Applied Biosystems) was used instead. The level of expression of the “house-keeping gene” cyclophilin was examined in each group and was shown not to be altered in the obese, diabetic or fasted state.

Primers used were as follows:

AGT-113:

| | |
|---|---|
| Forward primer: | |
| 5-CATGATGCCAGCCACCTG-3′ | [SEQ ID NO: 16] |
| Reverse primer: | |
| 5′-TCCCAAAGTAAATTAAACACATCAGAA-3′ | [SEQ ID NO: 17] |

Cyclophilin:

| | |
|---|---|
| Forward primer: | |
| 5′-CCC ACC GTG TTC TTC GAC AT-3′ | [SEQ ID NO: 18] |
| Reverse primer: | |
| 5′-CCA GTG CTC AGA GCA CGA AA-3′ | [SEQ ID NO: 19] |
| Probe: | |
| 5′-CGC GTC TCC TTC GAG CTG TTT | [SEQ ID NO: 20] |
| GC-3′ | |

The Psammomys obesus AGT-113 partial nucleotide sequence is as follows:

| | |
|---|---|
| TTTCATAGCTGGCATGATGCCAGCCACCTGGCAAAC | [SEQ ID NO: 2] |
| TGTGTCTCTTACCTGACTCCTTTCAAAATCAAGATA | |
| TTTTGAGAATAGTCTATATTCTGATGTGTTTAATTT | |
| ACTTTGGGAAGAAACTCCTTGCTTAAGTCTAAAATG | |
| GAAAACATTTTTTAATTAATAAAAAAAAAAA. | |

This sequence has some homology (84% homology over 65 nucleotides) to human clone RP11-368J13 (GenBank accession number AC008070).

The gene expression patterns strongly suggest that AGT-113 plays a role in body weight regulation and energy homeostasis through its actions in the hypothalamus. AGT-113 may also be involved in insulin's action or insulin resistance in the hypothalamus.

### EXAMPLE 10

### AGT-201

AGT-201 was determined to be differentially expressed in Psammomys obesus by membrane-based microarray (macroarray) analysis of the hypothalamus.

Primers used were as follows:—

AGT-201

| | |
|---|---|
| Forward primer: | |
| 5′-GCATGCCTGGTTGCCTG-3′ | [SEQ ID NO: 9] |
| Reverse primer: | |
| 5′-TTTCAAGATGGCCTGGCG-3′ | [SEQ ID NO: 10] |
| Probe: | |
| 5′-CCCTGGCAGGTGAGTTCATCAAGGC-3′ | [SEQ ID NO: 11] |

The partial nucleotide sequence of Psammomys obesus AGT-201 is as follows:

| | |
|---|---|
| CTTTAAGATT GGGANTNCGA TGATCTCTTG | [SEQ ID NO: 3] |
| GTGGCAGAGG TGGGAATCTC AGACTATGGT | |
| NCCAAGCTGA ACATGGAGCT NAGTNAAAAG | |
| TNCAAGCTGG TCAAAGAGGN CTACCCAGTG | |
| TTNTACCTCT TCCGAGACGG GGACTTTGAG | |
| AACCCAGTCC CATACAGTGG GGCAGTTAAG | |
| GTTGGAGCCA TCCAGCGCTG GCTAAAGGGG | |
| CAGGGGGTCT ACCTAGGCAT GCCTGGTTGC | |
| CTGCCTGCNT ACGATGCCCT GGCAGGTGAG | |
| TTCATCAAGG CCTCCAGTGT AGAGGCCCGC | |
| CAGGCCATCT TGAAAAAGGG GCAGGAAGGC | |
| CTCTCTGGTG TGAAGGAGAC TGNGAATAAG | |

Length:360 nucleotides

Analysis of the Psammomys obesus AGT-201 sequence showed high sequence homology with human AGT-201 (88%, X94910) and the rat homolog, Erp29 (89%, Y10264).

AGT-201 is known to be located on chromosome 12 and has been mapped to the interval D12S78-D12S79 (12q21-22). 2 animal QTLs are located in the vicinity of AGT-201, Qsbw and WeightI (Chagnon et al., Obes Res. 8(1): 89-117, 2000).

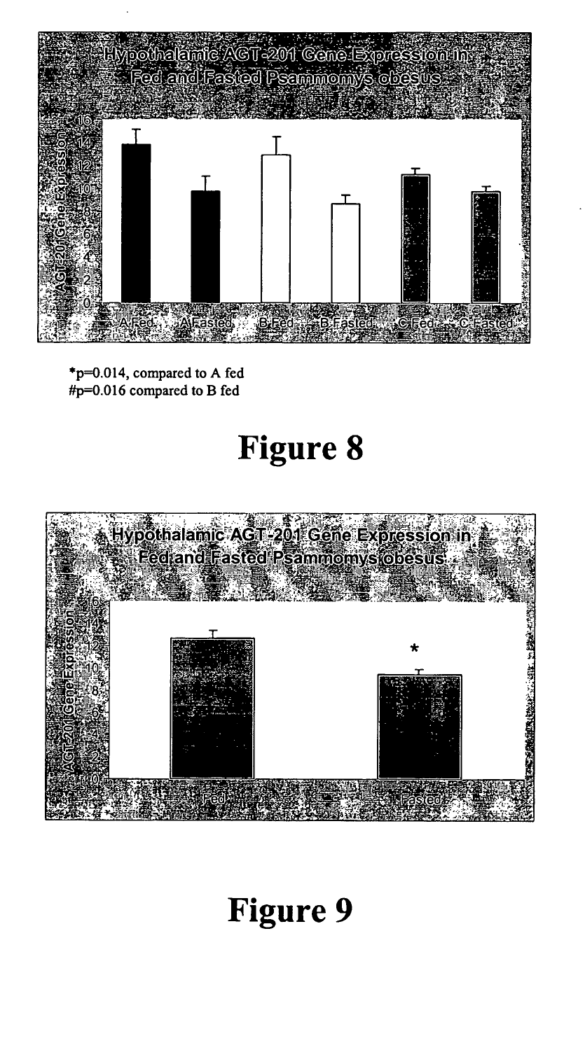

Across the three groups of animals, hypothalamic AGT-201 expression was decreased with fasting significantly in group A and B animals (FIG. 8) however there was no reduction of expression with fasting in group C animals. These results suggest a dysregulation of hypothalamic AGT-201 expression in diabetic group C animals.

There was significantly lower hypothalamic AGT-201 expression in the fasted animals compared to the fed animals (FIG. 9).

AGT-201 gene expression in the hypothalamus did not correlate with body weight, blood glucose or insulin concentration in the fed or fasted state.

These results suggest a role for AGT-201 in the central response to fasting and energy homeostasis, possibly by altering the protein expression, retention, retrieval or folding of secretory proteins exiting the endoplasmic reticulum. In addition, these results suggest that the role of AGT-201 in this regulation may be altered in the diabetic state thereby identifying this gene as a potential target for the development of diabetic treatments.

### EXAMPLE 11

### AGT-202

AGT-202 was determined to be differentially expressed in the hypothalamus of Psammomys obesus by macroarray analysis.

Primers used were as follows:—

AGT-202:

| | |
|---|---|
| Forward primer: | |
| 5′-CTGCAAAACGCCCATTCG-3′ | [SEQ ID NO: 14] |
| Reverse primer: | |
| 5′-TCATAGTCTCGCTCGCAGTAGG-3′ | [SEQ ID NO: 15] |

A portion of the Psammomys obesus AGT-202 sequence was obtained in order to design primers for gene expression studies.

| | |
|---|---|
| CCGGAGAGATCATGCACGCCCTCAAGATGACCTGGC | [SEQ ID NO: 4] |
| ACGTGCACTGNTTCACTTGTGCTGCCTGCAAAACGC | |
| CCATTCGCAACCGAGCGTTCTATATGGAGGAAGGGG | |
| CACCCTACTGCNAGCGAGACTATGAGAAGATGTTTG | |
| GCACAAAGTGCCGAGGCTGNGACTTCAAGATTGATG | |
| CTGGAGACCGCTTCCTGGAAGCGCTG. | |

Analysis of the Psammomys obesus AGT-202 sequence (154 nucleotides) showed high sequence homology with human AGT-202 (88%, L35246) and the rat homolog, AGT-202 (86%, AF096685).

AGT-202 is located on human chromosome 5. No QTL's relevant to obesity or diabetes were found.

AGT-202 gene expression appears to be widespread. cDNA sources include aorta, blood, brain, breast, colon, germ cell, kidney, larynx, lung, muscle, ovary, pancreas, pooled, prostate, stomach, testis, tonsil, uterus, whole embryo, brain, cervix, colon, eye, neck, kidney, lung, ovary, pancreas, prostate, tumor, skin, thymus, pooled, uterus, whole blood Immuno-localization studies in skeletal muscle using an anti-AGT-202 antibody showed that AGT-202 is present at the Z line and some transverse filaments in adult muscle sarcomeres.

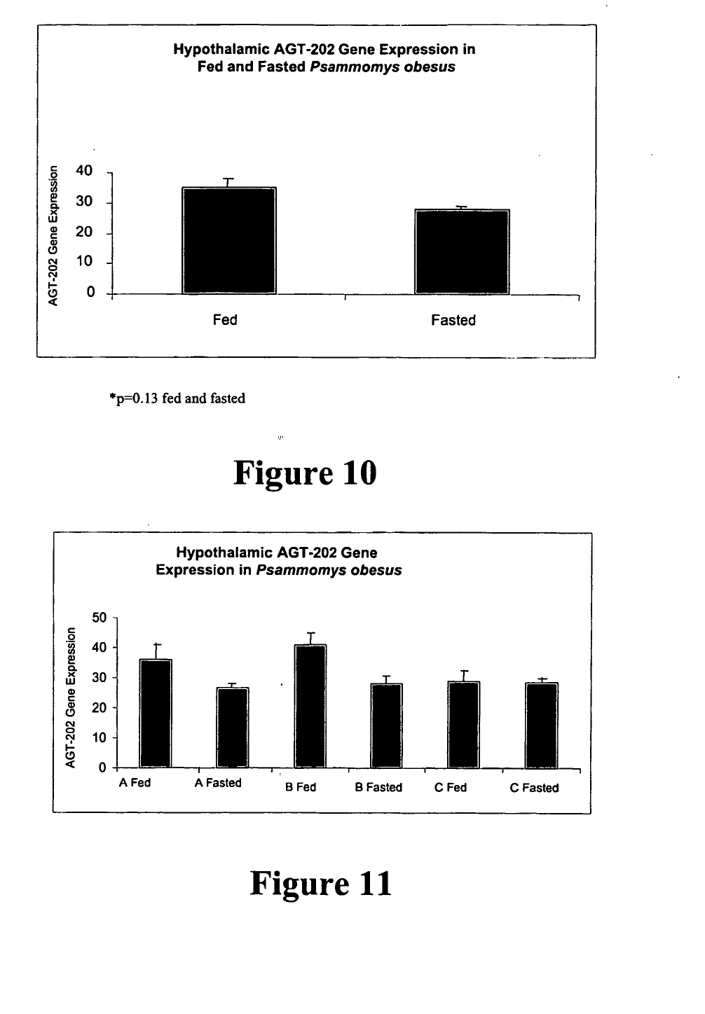

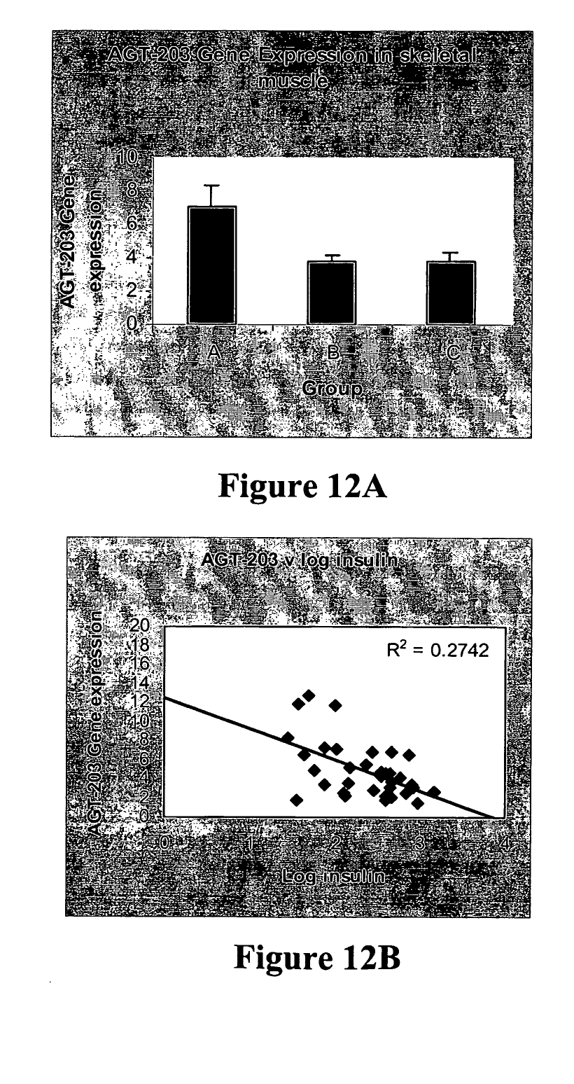

Overall, there was significantly greater AGT-202 gene expression in the hypothalamus of fed animals compared to the fasted animals (p=0.013) (FIG. 10). In addition, there appears to be greater AGT-202 gene expression in the hypothalamus of Group A and B fed animals compared to Group C animals, however, these differences were not significant (FIG. 11).

There was no correlation between AGT-202 gene expression and body weight, percent body fat or blood glucose or insulin levels.

### EXAMPLE 12

### Presenilins Interacting Rhomboid-Like Protease (A GT-203)

A human gene in the form of an expressed sequence tag (EST) with Accession Number AA131464 was found to be differentially expressed in skeletal muscle of lean, non-diabetic versus obese, diabetic animals by membrane-based microarray (macroarray). A human mRNA with a complete coding sequence that matched EST AA131464 was recently added to GenBank (on Nov. 1, 2000, Accession Number AF197937). The mRNA codes for a 379 amino acid protein they have named Presenilins interacting rhomboid-like protease (AGT-203).

Primers used were as follows:—

AGT-203:

| | |
|---|---|
| Forward primer: | |
| 5′-CCCACCTCTGGAAGAAACTGTCT-3′ | [SEQ ID NO: 6] |
| Reverse primer: | |
| 5′-CCTGTGAACCCAACAGTGAAGA-3′ | [SEQ ID NO: 7] |
| Probe: | |
| 5′-TTATCCTTCCCCCTACCCTATAAGAA | [SEQ ID NO: 8] |
| CTTTGGTG-3′ | |

A portion of the Psammomys obesus AGT-203 sequence was obtained in order to design primers for gene expression studies and is provided below:

[SEQ ID NO:5]

TGGAAGGTTGAACCTCGAAGATCAGACACAGGGTCAAGTGGTGAAGCTTA
CAAGAGAAGTGCCTTGATCCCACCTCTGGAAGAAACTGTCTTTTATCCTT
CCCCCTACCCTATAAGAACTTTGGTGAAGCCCTTTTTCTTCACTGTTGGG
TTCACAGGCTGTGCATTTGGATCAGCTGCCATTTGGCAATATGAATCACT
GAAATCCAGGGTCCAGAGTANNTGNNCGGAATGGCAGGAATGCCTGGACT
CAATGAAATCCAGGGTCC.

The AGT-203 gene appears to have a widespread tissue expression pattern. ESTs that correspond to the AGT-203 mRNA have been found in adrenal gland, blood, bone, brain, breast, colon, foreskin, germ cell, heart, kidney, lung, lymph, marrow, muscle, ovary, pancreas, parathyroid, placenta, prostate, skin, spleen, stomach, testis, tonsil, uterus and whole embryo.

The exon/intron structure of the human AGT-203 gene was deduced by aligning the mRNA sequence to human high throughput genome sequencing clones, then applying the GT-AG rule (where all introns start with GT and end with AG). The human AGT-203 gene has 10 exons. The first 4 exons were on clone RP 11-315J22 (Accession Number AC068644) which is from chromosome 3. The last 6 exons were on clone RP1′-637N15 (Accession Number AC020694) which is from chromosome 17. It is considered likely that one of the clones has been localized incorrectly. Studies are currently underway to determine which is the correct chromosome.

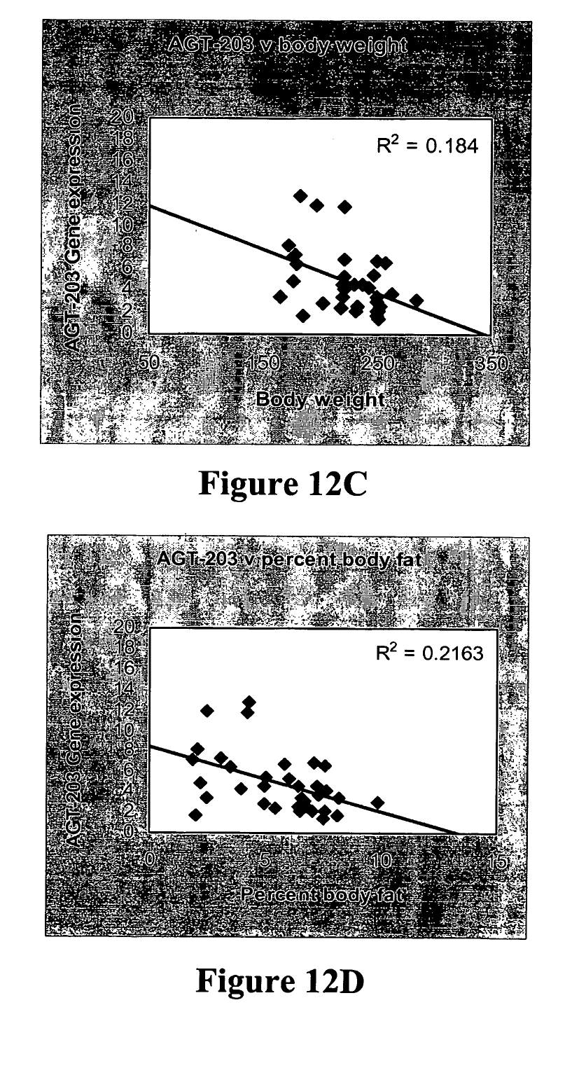

Gene expression studies by Taqman PCR (FIG. 12) showed a decrease in AGT-203 gene expression in skeletal muscle of obese, hyperinsulinemic group B and C animals when compared to lean group A animals (Group B p=0.014, Group C p=0.011). This relationship was further supported by a correlation between AGT-203 gene expression in skeletal muscle with log insulin (p=0.001), body weight (p=0.011) and percent body fat (p=0.006). There was no significant correlation with blood glucose levels.

AGT-203 gene expression was found to be reduced in the skeletal muscle of obese, hyperinsulinemic Psammomys obesus. A negative correlation was seen with body weight and plasma insulin levels. AGT-203 is thought to interact with presenilin proteins and be involved with protein cleavage.

The presenilins are involved in the proteolytic processing of transmembrane proteins such as APP and Notch. Although APP is most highly expressed in the central nervous system, it is ubiquitously expressed and its role in the skeletal muscle is not known. Diabetes is known to be a risk factor for Alzheimer's disease, and AGT-203 may play a role in both diseases. NOTCH is a membrane receptor and, after proteolytic processing within the membrane, has the ability to move to the nucleus and activate gene expression. AGT-203 may act through NOTCH to affect expression of genes involved in metabolism. Alternatively, the role of AGT-203 in obesity or diabetes may be through processing of another, as yet unidentified, transmembrane protein.

Other possible roles for presenilins include regulation of apoptosis and/or calcium homeostasis. Therefore there are a number of different pathways through which reduced AGT-203 expression could play a role in glucose or fat metabolism in the skeletal muscle, thereby affecting body weight and insulin action.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. An isolated nucleic acid molecule comprising a sequence of nucleotides encodings or complementary to a sequence encoding, a peptide molecule or derivative or homolog thereof,
wherein said nucleic acid molecule is differentially expressed hypothalamus tissue or muscle tissue either (a) in obese animals compared to lean animals, or (b) in fed animals compared to fasted animals,
wherein the nucleic acid molecule is selected from the group consisting of:
(i) a nucleic acid molecule comprising a nucleotide sequence selected from the group consisting of SEQ ID NOS: 1-5,
(ii) a nucleic acid molecule comprising a nucleotide sequence having at least about 90% identity thereto after optimal alignment, and
(iii) a nucleic acid molecule capable of hybridizing under high stringency conditions to a nucleotide sequence selected from the group consisting of SEQ ID NOS: 1-5 and a complement thereto.

2. The isolated nucleic acid molecule of claim 1 wherein the nucleic acid molecule comprises the nucleotide sequence set forth in SEQ ID NO:1.

3. The isolated nucleic acid molecule of claim 1 wherein the nucleic acid molecule comprises the nucleotide sequence set forth in SEQ ID NO:2.

4. The isolated nucleic acid molecule of claim 1 wherein the nucleic acid molecule comprises the nucleotide sequence set forth in SEQ ID NO:3.

5. The isolated nucleic acid molecule of claim 1 wherein the nucleic acid molecule comprises the nucleotide sequence set forth in SEQ ID NO:4.

6. The isolated nucleic acid molecule of claim 1 wherein the nucleic acid molecule comprises the nucleotide sequence set forth in SEQ ID NO:5.

7. An isolated protein molecule encoded by the nucleic acid molecule of claim 1.

8. An isolated protein of claim 7 encoded by a nucleotide sequence set forth in SEQ ID NO:1.

9. An isolated protein of claim 7 encoded by a nucleotide sequence set forth in SEQ ID NO:2.

10. An isolated protein of claim 7 encoded by a nucleotide sequence set forth in SEQ ID NO:3.

11. An isolated protein of claim 7 encoded by a nucleotide sequence set forth in SEQ ID NO:4.

12. An isolated protein of claim 7 encoded by a nucleotide sequence set forth in SEQ ID NO:5.

13. An isolated protein having at least about 90% identity to an amino acid sequence encoded by a nucleotide sequence selected from the group consisting of SEQ ID NOS:1-5after optimal alignments or a derivative, homolog, analog, chemical equivalent or mimetic of said protein.

14. A method for modulating expression of at least one nucleic acid molecule selected from the group consisting of AGT-106, AGT-113, AGT-201, AGT-202 and AGT-203 in a mammal, said method comprising
contacting at least one nucleic acid molecule selected from the group consisting of AGT-106, AGT-113, AGT-201, AGT-202 and AGT-203 with an effective amount of a modulator of at least one nucleic acid molecule selected from the group consisting of AGT-106, AGT-113, AGT-201, AGT-202 and AGT-203 expression for a time and under conditions sufficient to up-regulate or down-regulate or otherwise modulate expression of at least one nucleic acid molecule selected from the group consisting of AGT-106, AGT-113, AGT-201, AGT-202 and AGT-203.

15. A method of modulating activity of a protein selected from the group consisting of AGT-106, AGT-113, AGT-201, AGT-202 and AGT-203 in a mammal, said method comprising:
administering to said mammal a modulating effective amount of a molecule for a time and under conditions sufficient to increase or decrease the activity of a protein selected from the group consisting of AGT-106, AGT-113, AGT-201, AGT-202 and AGT-203.

16. A method of treating a mammal suffering from a condition characterized by at least one symptom selected from the group consisting of obesity, anorexia, diabetes and/or energy imbalance, said method comprising:
administering to said mammal an effective amount of an agent for a time and under conditions sufficient to modulate
(a) the expression of at least one nucleotide sequence selected from the group consisting of AGT-106, AGT-113, AGT-201, AGT-202 and AGT-203, or
(b) the activity of at least one protein selected from the group consisting of AGT-106, AGT-113, AGT-201, AGT-202 and/or AGT-203.

17. A method of treating a mammal suffering from a disease condition characterized by at least one symptom selected from the group consisting of obesity, anorexia, diabetes or energy imbalance, said method comprising:
administering to said mammal an effective amount of
(a) a protein molecule selected from the group consisting of AGT-106, AGT-113, AGT-201, AGT-202 and AGT-203,
(b) a derivative, homolog, analog, chemical equivalent or mimetic of the protein molecule as set forth in (a),
(c) a nucleic acid molecule selected from the group consisting of AGT-106, AGT-113, AGT-201, AGT-202 and AGT-203, or
(d) a derivative, homolog or analog of the nucleic acid molecule as set forth in (c).

18. (Canceled)

19. (Canceled)

20. (Canceled)

21. A composition comprising
(a) a modulator of
(i) the expression of a nucleic acid molecule selected from the group consisting of AGT-106, AGT-113, A GT-201, AGT-202 and AGT-203, or
(ii) the activity of a protein selected from the group consisting of AGT-106, AGT-113, AGT-201, AGT-202 and AGT-203 activity, and
(b) one or more pharmaceutically acceptable carriers or diluents.

22. A method for detecting a protein selected from the group consisting of AGT-106, AGT-113, AGT-201, AGT-202 and AGT-203 or a derivative or homolog thereof in a biological sample from a subject, said method comprising:
(a) contacting said biological sample with an antibody specific for
(i) a protein selected from the group consisting of AGT-106, AGT-113, AGT-201, AGT-202 and AGT-203, or
(ii) the antigenic derivatives or homologs for a time and under conditions sufficient for a complex to form, and
(b) then detecting said complex.

23. An isolated protein according to claim 13 wherein the protein is in a homodimeric form.

24. An isolated protein according to claim 13 wherein the protein is in a heterodimeric form.


SEQUENCE LISTING (20 sequences)

SEQ ID NO: 1 - DNA, 199 bp, Psammomys obesus
  misc_feature 1, 6, 9, 12: n = A,T,C or G
ntttgntgnccngctgtgtgtgttagaagaaaacagaaaaggaaagaaaaacaatcaccgcttttaagatagtttgtatcagcttagatttcatcatgactgttttacatactggaatttataaattgtaagttatcattttccaatgcgagccctttttaatgctttttaaaacttgtgaataaaattgatactcctt

SEQ ID NO: 2 - DNA, 175 bp, Psammomys obesus
tttcatagctggcatgatgccagccacctggcaaactgtgtctcttacctgactcctttcaaaatcaagatattttgagaatagtctatattctgatgtgtttaatttactttgggaagaaactccttgcttaagtctaaaatggaaaacattttttaattaataaaaaaaaaaa

SEQ ID NO: 3 - DNA, 360 bp, Psammomys obesus
  misc_feature 15, 17, 61, 81, 85, 92, 110, 123, 249, 353: n = A,T,C or G
ctttaagattgggantncgatgatctcttggtggcagaggtgggaatctcagactatggtnccaagctgaacatggagctnagtnaaaagtncaagctggtcaaagaggnctacccagtgttntacctcttccgagacggggactttgagaacccagtcccatacagtggggcagttaaggttggagccatccagcgctggctaaaggggcagggggtctacctaggcatgcctggttgcctgcctgcntacgatgccctggcaggtgagttcatcaaggcctccagtgtagaggcccgccaggccatcttgaaaaaggggcaggaaggcctctctggtgtgaaggagactgngaataag

SEQ ID NO: 4 - DNA, 206 bp, Psammomys obesus
  misc_feature 47, 120, 164: n = A,T,C or G
ccggagagatcatgcacgccctcaagatgacctggcacgtgcactgnttcacttgtgctgcctgcaaaacgcccattcgcaaccgagcgttctatatggaggaaggggcaccctactgcnagcgagactatgagaagatgtttggcacaaagtgccgaggctgngacttcaagattgatgctggagaccgcttcctggaagcgctg

SEQ ID NO: 5 - DNA, 268 bp, Psammomys obesus
  misc_feature 221, 222, 225, 226: n = A,T,C or G
tggaaggttgaacctcgaagatcagacacagggtcaagtggtgaagcttacaagagaagtgccttgatcccacctctggaagaaactgtcttttatccttccccctaccctataagaactttggtgaagccctttttcttcactgttgggttcacaggctgtgcatttggatcagctgccatttggcaatatgaatcactgaaatccagggtccagagtanntgnncggaatggcaggaatgcctggactcaatgaaatccagggtcc

SEQ ID NO: 6 - DNA, 23 bp, Artificial Sequence
  Forward primer (AGT-203)
cccacctctggaagaaactgtct

SEQ ID NO: 7 - DNA, 22 bp, Artificial Sequence
  Reverse primer (AGT-203)
cctgtgaacccaacagtgaaga

SEQ ID NO: 8 - DNA, 34 bp, Artificial Sequence
  Probe (AGT-203)
ttatccttccccctaccctataagaactttggtg

SEQ ID NO: 9 - DNA, 17 bp, Artificial Sequence
  Forward primer (AGT-201)
gcatgcctggttgcctg

SEQ ID NO: 10 - DNA, 18 bp, Artificial Sequence
  Reverse primer (AGT-201)
tttcaagatggcctggcg

SEQ ID NO: 11 - DNA, 25 bp, Artificial Sequence
  Probe (AGT-201)
ccctggcaggtgagttcatcaaggc

SEQ ID NO: 12 - DNA, 26 bp, Artificial Sequence
  Forward primer (AGT-106)
caatcaccgcttttaagatagtttgt

SEQ ID NO: 13 - DNA, 20 bp, Artificial Sequence
  Reverse primer (AGT-106)
agcattaaaaagggctcgca

SEQ ID NO: 14 - DNA, 18 bp, Artificial Sequence
  Forward primer (AGT-202)
ctgcaaaacgcccattcg

SEQ ID NO: 15 - DNA, 22 bp, Artificial Sequence
  Reverse primer (AGT-202)
tcatagtctcgctcgcagtagg

SEQ ID NO: 16 - DNA, 18 bp, Artificial Sequence
  Forward primer (AGT-113)
catgatgccagccacctg

SEQ ID NO: 17 - DNA, 27 bp, Artificial Sequence
  Reverse primer (AGT-113)
tcccaaagtaaattaaacacatcagaa

SEQ ID NO: 18 - DNA, 20 bp, Artificial Sequence
  Forward primer (Cyclophilin)
cccaccgtgttcttcgacat

SEQ ID NO: 19 - DNA, 20 bp, Artificial Sequence
  Reverse primer (Cyclophilin)
ccagtgctcagagcacgaaa

SEQ ID NO: 20 - DNA, 23 bp, Artificial Sequence
  Probe (Cyclophilin)
cgcgtctccttcgagctgtttgc